(19) United States
(12) Patent Application Publication
(10) Pub. No.: US 2017/0290775 A1
(43) Pub. Date: Oct. 12, 2017

(54) MICRONIZED DELIVERY MATERIAL AND METHODS FOR MANUFACTURING THEREOF

(71) Applicant: Therakine Bio Delivery GmbH, Berlin (DE)

(72) Inventors: Andreas VOIGT, Berlin (DE); Mariana DOBRANIS, Berlin (DE); Sonja LEHMANN, Berlin (DE); Christoph DUNMANN, Berlin (DE)

(21) Appl. No.: 15/516,686

(22) PCT Filed: Oct. 6, 2015
(86) PCT No.: PCT/US2015/054249

§ 371 Date: Apr. 4, 2017

Related U.S. Application Data
(60) Provisional application No. 62/060,642, filed on Oct. 7, 2014.

(51) Int. Cl. A61K 9/14; A61K 47/12; A61K 9/00; A61K 38/00; A61K 47/10

(52) U.S. Cl. CPC A61K 9/145; A61K 38/00; A61K 47/10; A61K 9/0024; A61K 47/12

(57) ABSTRACT

The present invention discloses methods for manufacturing drug delivery composition comprising at least one pharmaceutically active compound, at least one anti-caking agent and at least one oil. The anti-caking agent and the oil form the matrix when mixed, which then embeds the pharmaceutically active compound.

### CROSS-REFERENCE TO RELATED APPLICATIONS

The present application is a national stage entry according to 35 U.S.C. §371 of PCT Application No. PCT/US15/54249 filed on Oct. 6, 2015, which claims priority to U.S. Provisional Application Ser. No. 62/060,642 filed on Oct. 7, 2014.

### BACKGROUND OF THE INVENTION

### Field of the Invention

Most therapeutic dosage forms include mixtures of one or more pharmaceutically active compounds with additional components referred to as excipients. The pharmaceutically active compounds are substances that exert a pharmacological effect on a living tissue or organism, whether used for prevention, treatment, or cure of a disease. The pharmaceutically active compounds can be naturally occurring or synthetic substances, or can be produced by recombinant methods, or any combination of these approaches.

Various embodiments relate generally to the field of controlled drug release, particularly to methods for manufacturing drug-delivery compositions and the use of a pharmaceutically acceptable, injectable sustained delivery system for biologicals, such as therapeutic proteins.

### Background

Most therapeutic dosage forms include mixtures of one or more pharmaceutically active compounds with additional components referred to as excipients. The pharmaceutically active compounds are substances that exert a pharmacological effect on a living tissue or organism, whether used for prevention, treatment, or cure of a disease. These compounds can be naturally occurring or synthetic substances, or can be produced by recombinant methods, or any combination of these approaches.

Numerous methods have been devised for delivering the pharmaceutically active compounds into living organisms, each with more or less success. Traditional oral therapeutic dosage forms include both solids (tablets, capsules, pills, etc.) and liquids (solutions, suspensions, emulsions, etc.). Parenteral dosage forms include solids, liquids, aerosols (administered by inhalers, etc.), injectables (administered with syringes, micro-needle arrays, etc.), topicals (foams, ointments, etc.), and suppositories, among other dosage forms. Although these dosage forms might be effective in delivering low molecular weight pharmaceutically active compounds, each of these various methods suffers from one or more drawbacks, including the lack of bioavailability as well as the inability to completely control either the spatial or the temporal component of the pharmaceutically active com distribution when it comes to high molecular weight pharmaceutically active compounds. These drawbacks are especially challenging for administering biotherapeutics, i.e. pharmaceutically active peptides (e.g. growth factors), proteins (e.g. enzymes, antibodies), oligonucleotides and nucleic acids (e.g. RNA, DNA, PNA, aptamers, spiegelmers), hormones and other natural substances or synthetic substances mimicking such, since many types of pharmacologically active biomolecules are at least partially broken down either in the digestive tract or in the blood system and are subsequently delivered in suboptimal doses to the target site.

Therefore, an ongoing need exists for improved drug-delivery methods in the life sciences, including, but not limited to, human and veterinary medicine. One important goal for any new drug-delivery method is to deliver the desired therapeutic agent(s) to a specific place in the body over a specific and controllable period of time, i.e. controlling the delivery of one or more substances to specific organs and tissues in the body in both a spatial and temporal manner. Methods for accomplishing this spatially and temporally controlled delivery are known as controlled-release drug-delivery methods. Delivering the pharmaceutically active compounds to specific organs and tissues in the body offers several potential advantages, including increased patient compliance, extending activity, lowering the required dose, minimizing side effects, and permitting the use of more potent therapeutics. In some cases, controlled-release drug-delivery methods can even allow the administration of therapeutic agents which would otherwise be too toxic or ineffective for use.

There are five broad types of solid dosage forms for controlled-delivery oral administration: reservoir and matrix diffusive dissolution, osmotic, ion-exchange resins, and prodrugs. For parenteral, most of the above solid dosage forms are available, as well as injections (intravenous, intramuscular, etc.), transdermal systems, and implants. Numerous products have been developed for both oral and parenteral administration, including depots, pumps, and micro- and nano-particles.

The incorporation of the pharmaceutically active compounds into polymer matrices acting as a core reservoir is one approach for controlling their delivery. Contemporary approaches for formulating such drug-delivery systems are dependent on technological capabilities as well as the specific requirements of the application. For sustained delivery systems there a two main structural approaches: the release controlled by diffusion through a barrier, such as shell, coat, or membrane, and the release controlled by the intrinsic local binding strength of the pharmaceutically active compound(s) to the core or to other ingredients in the core reservoir. Thus, there is a significant and unmet need for improving drug-delivery methods and compositions. The present embodiments satisfy this long standing need in the art.

### SUMMARY

Certain representative embodiments are described herein, and do not limit the scope of the embodiments in any way.

In a non-limiting embodiment, a method for manufacturing a drug-delivery composition is described. In another embodiment, this method comprises dissolving at least one pharmaceutically active compound in an aqueous solution, mixing the pharmaceutically active compound dissolved in the aqueous solution with at least one anti-caking agent and oil to form a matrix that embeds the pharmaceutically active compound, adding excess of at least one of the anti-caking agent to the matrix, and mixing the matrix and the embedded pharmaceutically active compound by continuously pressing and folding the matrix and the embedded pharmaceutically active compound, where the mixing disperses the pharmaceutically active compound uniformly and transforms the matrix into a microparticulate form.

In another embodiment, the pharmaceutically active compound comprises a compound selected from the group consisting of a protein, a humanized monoclonal antibody, a human monoclonal antibody, a chimeric antibody, an immunoglobulin, fragment, derivative or fraction thereof, a synthetic, semi-synthetic or biosynthetic substance mimicking immunoglobulins or fractions thereof, an antigen binding protein or fragment thereof, a fusion protein or peptide or fragment thereof, a receptor antagonist, an antiangiogenic compound, an intracellular signaling inhibitor, a peptide with a molecular mass equal to or higher than 3 kDa, a ribonucleic acid (RNA), a deoxyribonucleic acid (DNA), a plasmid, a peptide nucleic acid (PNA), a steroid, a corticosteroid, an adrenocorticostatic, an antibiotic, an antidepressant, an antimycotic, a [beta]-adrenolytic, an androgen or antiandrogen, an antianemic, an anabolic, an anesthetic, an analeptic, an antiallergic, an antiarrhythmic, an antiarterosclerotic, an antibiotic, an antifibrinolytic, an anticonvulsive, an anti-inflammatory drug, an anticholinergic, an antihistamine, an antihypertensive, an antihypotensive, an anticoagulant, an antiseptic, an antihemorrhagic, an antimyasthenic, an antiphlogistic, an antipyretic, a beta-receptor antagonist, a calcium channel antagonist, a cell, a cell differentiation factor, a chemokine, a chemotherapeutic, a co-enzyme, a cytotoxic agent, a prodrug of a cytotoxic agent, a cytostatic, an enzyme and its synthetic or biosynthetic analogue, a glucocorticoid, a growth factor, a hemostatic, a hormone and its synthetic or biosynthetic analogue, an immunosuppressant, an immunostimulant, a mitogen, a physiological or pharmacological inhibitor of mitogens, a mineralocorticoid, a muscle relaxant, a narcotic, a neurotransmitter, a precursor of neurotransmitter, an oligonucleotide, a peptide, a (para)-sympathomimetic, a (para)-sympatholytic, a sedating agent, a spasmolytic, a vasoconstrictor, a vasodilator, a vector, a virus, a virus-like particle, a virustatic, a wound healing substance and a combination thereof.

In yet another embodiment, the pharmaceutically active compound is either alone or in combination with at least one excipient. In still yet another embodiment, the excipient is selected from the group consisting of monosaccharides, disaccharides, oligosaccharides, polysaccharides, hyaluronic acid, pectin, gum arabic and other gums, albumin, chitosan, collagen, collagen-n-hydroxysuccinimide, fibrin, fibrinogen, gelatin, globulin, polyaminoacids, polyurethane comprising amino acids, prolamin, protein-based polymers, copolymers and derivatives thereof, and mixtures thereof. In another embodiment, the mixing by repeated pressing and folding comprises repeated cycles of pressing and folding in an algorithmic manner of the matrix and the pharmaceutically active ingredient. In yet another embodiment, the mixing by repeated pressing and folding allows water in the matrix to evaporate. In still yet another embodiment, the pressing comprises applying a pressure of not more than 10⁶ N·m⁻². In another embodiment, the anti-caking agent is a compound selected from the group consisting of magnesium stearate, magnesium palmitate and similar compounds. In still yet another embodiment, the oil is selected from the group consisting of plant oil, castor oil, jojoba oil, soybean oil, silicon oils, paraffin oils and mineral oils, and oxethylated plant oils. In another embodiment, the final matrix comprises about ten percent of the pharmaceutically active compound, about ninety percent of the anti-caking agent and about ten percent of the oil. In yet another embodiment, the anti-caking agent, the oil and the pharmaceutically active compound are biodegradable and biocompatible.

In another non-limiting embodiment, a method for manufacturing a drug-delivery composition is disclosed. In another embodiment, this method comprises mixing at least one anti-caking agent and at least one oil, where fraction of the oil is greater than final concentration of the ready-for-application delivery mix, adding excess of the anti-caking agent to the mixture comprising the anti-caking agent and the oil, mixing the anti-caking agent and oil mixture by continuously pressing and folding the mixture to transform the mixture into a microparticulate matrix, dissolving at least one pharmaceutically active compound in an aqueous solution, adding the pharmaceutically active compound dissolved in the aqueous solution to the microparticulate matrix, and dispersing the pharmaceutically active compound dissolved in the aqueous solution within the matrix by continuously pressing and folding the matrix.

In another embodiment, the pharmaceutically active compound comprises a compound selected from the group consisting of a protein, a humanized monoclonal antibody, a human monoclonal antibody, a chimeric antibody, an immunoglobulin, fragment, derivative or fraction thereof, a synthetic, semi-synthetic or biosynthetic substance mimicking immunoglobulins or fractions thereof, an antigen binding protein or fragment thereof, a fusion protein or peptide or fragment thereof, a receptor antagonist, an antiangiogenic compound, an intracellular signaling inhibitor, a peptide with a molecular mass equal to or higher than 3 kDa, a ribonucleic acid (RNA), a deoxyribonucleic acid (DNA), a plasmid, a peptide nucleic acid (PNA), a steroid, a corticosteroid, an adrenocorticostatic, an antibiotic, an antidepressant, an antimycotic, a [beta]-adrenolytic, an androgen or antiandrogen, an antianemic, an anabolic, an anesthetic, an analeptic, an antiallergic, an antiarrhythmic, an antiarterosclerotic, an antibiotic, an antifibrinolytic, an anticonvulsive, an anti-inflammatory drug, an anticholinergic, an antihistamine, an antihypertensive, an antihypotensive, an anticoagulant, an antiseptic, an antihemorrhagic, an antimyasthenic, an antiphlogistic, an antipyretic, a beta-receptor antagonist, a calcium channel antagonist, a cell, a cell differentiation factor, a chemokine, a chemotherapeutic, a coenzyme, a cytotoxic agent, a prodrug of a cytotoxic agent, a cytostatic, an enzyme and its synthetic or biosynthetic analogue, a glucocorticoid, a growth factor, a hemostatic, a hormone and its synthetic or biosynthetic analogue, an immunosuppressant, an immunostimulant, a mitogen, a physiological or pharmacological inhibitor of mitogens, a mineralocorticoid, a muscle relaxant, a narcotic, a neurotransmitter, a precursor of neurotransmitter, an oligonucleotide, a peptide, a (para)-sympathomimetic, a (para)-sympatholytic, a sedating agent, a spasmolytic, a vasoconstrictor, a vasodilator, a vector, a virus, a virus-like particle, a virustatic, a wound healing substance and a combination thereof.

In yet another embodiment, the pharmaceutically active compound is either alone or in combination with at least one excipient. In still yet another embodiment, the excipient is selected from the group consisting of monosaccharides, disaccharides, oligosaccharides, polysaccharides, hyaluronic acid, pectin, gum arabic and other gums, albumin, chitosan, collagen, collagen-n-hydroxysuccinimide, fibrin, fibrinogen, gelatin, globulin, polyaminoacids, polyurethane comprising amino acids, prolamin, protein-based polymers, copolymers and derivatives thereof, and mixtures thereof. In still yet another embodiment, the mixing by repeated pressing and folding comprises repeated cycles of pressing and folding in an algorithmic manner of the matrix and the pharmaceutically active ingredient. In another embodiment, the mixing by repeated pressing and folding allows water in the matrix to evaporate. In yet another embodiment, the pressing comprises applying a pressure of not more than 10⁶ N·m⁻². In another embodiment, the anti-caking agent is a compound selected from the group consisting of magnesium stearate, magnesium palmitate and similar compounds. In still yet another embodiment, the oil is selected from the group consisting of plant oil, castor oil, jojoba oil, soybean oil, silicon oils, paraffin oils and mineral oils, and oxethylated plant oils. In another embodiment, the final matrix comprises about ten percent weight by volume (w/v) of pharmaceutically active compound, in a matrix comprising about ninety percent w/v of anti-caking agent and about ten percent w/v of oil. In yet another embodiment, the anti-caking agent, the oil and the pharmaceutically active compound are biodegradable and biocompatible.

In another non-limiting embodiment, the method for manufacturing a drug-delivery composition is disclosed. In another embodiment, this method comprises dissolving at least one pharmaceutically active compound in an aqueous solution, mixing the pharmaceutically active compound dissolved in the aqueous solution with at least one anti-caking agent, at least one oil and water to form a matrix that embeds the pharmaceutically active compound, and mixing the matrix and the embedded pharmaceutically active compound further until the mixture is transformed into a microparticulate form.

In yet another embodiment, the pharmaceutically active compound comprises a compound selected from the group consisting of a protein, a humanized monoclonal antibody, a human monoclonal antibody, a chimeric antibody, an immunoglobulin, fragment, derivative or fraction thereof, a synthetic, semi-synthetic or biosynthetic substance mimicking immunoglobulins or fractions thereof, an antigen binding protein or fragment thereof, a fusion protein or peptide or fragment thereof, a receptor antagonist, an antiangiogenic compound, an intracellular signaling inhibitor, a peptide with a molecular mass equal to or higher than 3 kDa, a ribonucleic acid (RNA), a deoxyribonucleic acid (DNA), a plasmid, a peptide nucleic acid (PNA), a steroid, a corticosteroid, an adrenocorticostatic, an antibiotic, an antidepressant, an antimycotic, a [beta]-adrenolytic, an androgen or antiandrogen, an antianemic, an anabolic, an anesthetic, an analeptic, an antiallergic, an antiarrhythmic, an antiarterosclerotic, an antibiotic, an antifibrinolytic, an anticonvulsive, an anti-inflammatory drug, an anticholinergic, an antihistamine, an antihypertensive, an antihypotensive, an anticoagulant, an antiseptic, an antihemorrhagic, an antimyasthenic, an antiphlogistic, an antipyretic, a beta-receptor antagonist, a calcium channel antagonist, a cell, a cell differentiation factor, a chemokine, a chemotherapeutic, a coenzyme, a cytotoxic agent, a prodrug of a cytotoxic agent, a cytostatic, an enzyme and its synthetic or biosynthetic analogue, a glucocorticoid, a growth factor, a hemostatic, a hormone and its synthetic or biosynthetic analogue, an immunosuppressant, an immunostimulant, a mitogen, a physiological or pharmacological inhibitor of mitogens, a mineralocorticoid, a muscle relaxant, a narcotic, a neurotransmitter, a precursor of neurotransmitter, an oligonucleotide, a peptide, a (para)-sympathomimetic, a (para)-sympatholytic, a sedating agent, a spasmolytic, a vasoconstrictor, a vasodilator, a vector, a virus, a virus-like particle, a virustatic, a wound healing substance and a combination thereof.

In yet another embodiment, the pharmaceutically active compound is either alone or in combination with at least one excipient. In still yet another embodiment, the excipient is selected from the group consisting of monosaccharides, disaccharides, oligosaccharides, polysaccharides, hyaluronic acid, pectin, gum arabic and other gums, albumin, chitosan, collagen, collagen-n-hydroxysuccinimide, fibrin, fibrinogen, gelatin, globulin, polyaminoacids, polyurethane comprising amino acids, prolamin, protein-based polymers, copolymers and derivatives thereof, and mixtures thereof. In another embodiment, the mixing of the matrix and the embedded pharmaceutically active compound comprises repeated cycles of pressing and folding in an algorithmic manner of the matrix and the pharmaceutically active ingredient. In yet another embodiment, the mixing by repeated pressing and folding allows water in the matrix to evaporate. In still yet another embodiment, the pressing comprises applying a pressure of not more than 10⁶N·m⁻².

In another embodiment, the anti-caking agent is a compound selected from the group consisting of magnesium stearate, magnesium palmitate and similar compounds. In still yet another embodiment, the oil is selected from the group consisting of plant oil, castor oil, jojoba oil, soybean oil, silicon oils, paraffin oils and mineral oils, and oxethylated plant oils. In another embodiment, the final matrix comprises about ten percent weight by volume (w/v) of the pharmaceutically active compound, in a matrix comprising about ninety percent w/v of the anti-caking agent and about ten percent w/v of the oil. In yet another embodiment, the anti-caking agent, the oil and the pharmaceutically active compound are biodegradable and biocompatible.

In another non-limiting embodiment, the method for manufacturing a drug-delivery composition is disclosed. In another embodiment, this method comprises mixing at least one anti-caking agent and at least one oil until a powder-like matrix is formed, where amount of the anti-caking agent and the oil in the mixture is in a ratio of final concentration of the anti-caking agent and the oil where the ratio is between about 95%:5% and about 80%:20%, dissolving at least one pharmaceutically active compound in an aqueous solution, adding the pharmaceutically active compound dissolved in the aqueous solution to the matrix, and mixing the pharmaceutically active compound with the matrix by continuously pressing and folding the matrix and the embedded pharmaceutically active compound, where the mixing disperses the pharmaceutically active compound uniformly and transforms the matrix into a microparticulate form.

In another embodiment, the pharmaceutically active compound comprises a compound selected from the group consisting of a protein, a humanized monoclonal antibody, a human monoclonal antibody, a chimeric antibody, an immunoglobulin, fragment, derivative or fraction thereof, a synthetic, semi-synthetic or biosynthetic substance mimicking immunoglobulins or fractions thereof, an antigen binding protein or fragment thereof, a fusion protein or peptide or fragment thereof, a receptor antagonist, an antiangiogenic compound, an intracellular signaling inhibitor, a peptide with a molecular mass equal to or higher than 3 kDa, a ribonucleic acid (RNA), a deoxyribonucleic acid (DNA), a plasmid, a peptide nucleic acid (PNA), a steroid, a corticosteroid, an adrenocorticostatic, an antibiotic, an antidepressant, an antimycotic, a [beta]-adrenolytic, an androgen or antiandrogen, an antianemic, an anabolic, an anesthetic, an analeptic, an antiallergic, an antiarrhythmic, an antiarterosclerotic, an antibiotic, an antifibrinolytic, an anticonvulsive, an anti-inflammatory drug, an anticholinergic, an antihistamine, an antihypertensive, an antihypotensive, an anticoagulant, an antiseptic, an antihemorrhagic, an antimyasthenic, an antiphlogistic, an antipyretic, a beta-receptor antagonist, a calcium channel antagonist, a cell, a cell differentiation factor, a chemokine, a chemotherapeutic, a coenzyme, a cytotoxic agent, a prodrug of a cytotoxic agent, a cytostatic, an enzyme and its synthetic or biosynthetic analogue, a glucocorticoid, a growth factor, a hemostatic, a hormone and its synthetic or biosynthetic analogue, an immunosuppressant, an immunostimulant, a mitogen, a physiological or pharmacological inhibitor of mitogens, a mineralocorticoid, a muscle relaxant, a narcotic, a neurotransmitter, a precursor of neurotransmitter, an oligonucleotide, a peptide, a (para)-sympathomimetic, a (para)-sympatholytic, a sedating agent, a spasmolytic, a vasoconstrictor, a vasodilator, a vector, a virus, a virus-like particle, a virustatic, a wound healing substance and a combination thereof.

In yet another embodiment, the pharmaceutically active compound is either alone or in combination with at least one excipient. In still yet another embodiment, the excipient is selected from the group consisting of monosaccharides, disaccharides, oligosaccharides, polysaccharides, hyaluronic acid, pectin, gum arabic and other gums, albumin, chitosan, collagen, collagen-n-hydroxysuccinimide, fibrin, fibrinogen, gelatin, globulin, polyaminoacids, polyurethane comprising amino acids, prolamin, protein-based polymers, copolymers and derivatives thereof, and mixtures thereof. In another embodiment, the mixing by repeated pressing and folding comprises repeated cycles of pressing and folding in an algorithmic manner of the matrix and the pharmaceutically active ingredient. In yet another embodiment, the mixing by repeated pressing and folding allows water in the matrix to evaporate. In still yet another embodiment, the pressing comprises applying a pressure of not more than 10⁶N·m⁻².

In another embodiment, the anti-caking agent is a compound selected from the group consisting of magnesium stearate, magnesium palmitate and similar compounds. In yet another embodiment, the oil is selected from the group consisting of plant oil, castor oil, jojoba oil, soybean oil, silicon oils, paraffin oils and mineral oils, and oxethylated plant oils. In still yet another embodiment, the final concentration of the pharmaceutically active compound, the anti-caking agent and the oil in the matrix comprises about ten percent weight by volume (w/v) of pharmaceutically active compound, in a matrix comprising about ninety percent w/v of anti-caking agent and about ten percent w/v of oil. In another embodiment, the anti-caking agent, the oil and the pharmaceutically active compound are biodegradable and biocompatible.

Those skilled in the art will recognize additional features and advantages upon reading the following detailed description, and upon viewing the accompanying drawings.

### BRIEF DESCRIPTION OF THE DRAWINGS

FIGS. 1A-1B show release profile of the one-step process of manufacturing the drug delivery composition. FIG. 1A shows sustained release of the pharmaceutically active compound related to the percentage of original payload of the pharmaceutically active compound. FIG. 1B shows the release profile when the release is normalized to a 10 mg matrix system appropriate for intravitreal injection.

FIGS. 2A-2B show release profile of the two-step process of manufacturing the drug delivery composition. FIG. 2A shows sustained release of the pharmaceutically active compound related to percentage of original payload of pharmaceutically active compound. FIG. 2B shows the release profile when the release is normalized to 10 mg matrix system appropriate for intravitreal injection.

### DETAILED DESCRIPTION

The following language and descriptions of certain embodiments are provided. However, it will be understood that no limitations of the present embodiments are intended, and that further alterations, modifications, and applications of the principles of the present embodiments are also included.

As used herein, the term “matrix” refers to a material in which the pharmaceutically active compound is embedded. For instance, the anti-caking agent and oil are mixed together as described in the various embodiments to form a matrix that enables the pharmaceutically active compound to be embedded within.

As used herein, the term “oil” refers to a neutral, nonpolar chemical substance that is a viscous liquid at ambient temperatures. Some examples of oil that can be used in the different embodiments of the present embodiments include but are not limited to plant oil, castor oil, jojoba oil, soybean oil, silicon oils, paraffin oils and mineral oils, and oxethylated plant oils.

As used herein, the term “micronization” is understood in the art to refer to a process that reduces size of the particles of the pharmaceutically active compound so that these can be efficiently embedded within the matrix. There are different ways by which micronization can be performed. Some examples include but are not limited to using a fluid energy or mechanical means.

As used herein, the term “pharmaceutically active compound” refers to a compound or combination of compounds that are used in manufacturing a drug product. This compound may also have a direct effect on the disease diagnosis, prevention, treatment or cure. Some examples of the pharmaceutically active compound that can be used herein are listed supra.

As used herein, the term “receptor antagonist” refers to a type receptor specific ligand or drug that can block receptor-mediated response by binding to the receptor and preventing the binding of agonists to the receptor. Some examples of such receptor antagonist include but are not limited to anti-TNF alpha, anti-Interleukin-1, anti-Interleukin-6, anti-epidermal growth factor receptor, anti-dopamine receptor, anti-Angiotensin II receptor, anti-aldosterone receptor and anti-leukotriene receptor.

As used herein, the term “anti-angiogenic compounds” refer to compounds that inhibit the growth of new blood vessels, reduce the production of pro-angiogenic factors, prevent the pro-angiogenic factors from binding to their receptors, block the actions of pro-angiogenic factors or a combination thereof. Some examples of these compounds include but are not limited to compounds that inhibit the activity of VEGF, PDGF, and angiogenesis stimulators.

As used herein, the term “intracellular signaling inhibitors” refer to compounds that block signaling pathways by blocking the binding of ligands to the receptor involved in cell signaling or signal transduction, the actions of the receptors or the combination thereof. These compounds are useful in treatment, prevention, diagnosis or cure of various diseases. Some examples of intracellular signaling inhibitors include but are not limited to JAK1, JAK3 and SYK.

As used herein, the term “sustained release” refers to a dosage form designed to release a drug at a predetermined rate in order to maintain a constant drug concentration in the system for a specific period of time.

As used herein, the term “anti-caking agent” refers to an additive placed in powdered or granulated material to prevent the formation of lumps. Some examples of anti-caking agents include but are not limited to tricalcium phosphate, powdered cellulose, magnesium stearate, sodium bicarbonate, sodium ferrocyanide, potassium ferrocyanide, calcium ferrocyanide, bone phosphate, sodium silicate, silicon dioxide, calcium silicate, magnesium trisilicate, talcum powder, sodium aluminosilicate, potassium aluminium silicate, calcium aluminosilicate, bentonite, aluminium silicate, stearic acid and polydimethylsiloxane.

As used herein, the term “microparticulate” refers to small, drug-containing low-molecular weight particles that are suspended in a liquid carrier medium.

As used herein, the term “intravitreal application” refers to one of the routes of administration of a drug or other substance, wherein the drug or other substance is delivered into the vitreous, near the retina at the back of the eye. The vitreous is a jelly-like fluid that fills the inside of the eye.

The present embodiment discloses methods for manufacturing a drug-delivery composition. In general, these methods comprise mixing at least three of the following ingredients: (1) a pharmaceutically active compound dissolved in an aqueous solution, (2) an anti-caking agent, for example, magnesium stearate; and (3) oil. The sequence in which these ingredients are mixed and the amounts of these ingredients may differ in the different embodiments.

In one embodiment, there is a method of manufacturing a drug-delivery composition that comprises at least one pharmaceutically active compound in an aqueous solution and then mixing it with at least one anti-caking agent, such as magnesium stearate and at least one oil. An excess amount of the anti-caking agent is added and this is followed by continuous mixing till the pharmaceutically active compound disperse uniformly within the matrix that is formed by mixing the anti-caking agent and oil. In another embodiment, the method of manufacturing a drug-delivery composition comprises mixing at least one anti-caking agent and at least one first, where the fraction of oil is greater than final concentration of the injectable micronized matrix. An excess of anti-caking agent is added to this mixture followed by adding pharmaceutically active compound dissolved in an aqueous solution to the mixture. The entire mixture is mixed thoroughly until the pharmaceutically active compound disperses within the matrix.

In yet another embodiment, the method of manufacturing the drug-delivery composition comprises dissolving at least one pharmaceutically active compound in an aqueous solution and then mixing this with at least one anti-caking agent, at least one oil and water such that the active pharmaceutical agent is embedded within the matrix formed by mixing the anti-caking agent, oil and water. The mixing is continued until the mixture is transformed into a microparticulate form. In still yet another embodiment, the method of manufacturing a drug-delivery composition comprises mixing at least one anti-caking agent and at least one oil until a powder-like matrix is formed. The amount of the anti-caking agent and oil is in a ratio of the final concentration of the anti-caking agent and oil. The pharmaceutically active compound dissolved in the aqueous solution is then added to the matrix formed by mixing the anti-caking agent and oil and the entire mixture is then mixed continuously until the pharmaceutically active compound disperse throughout the matrix uniformly and the matrix is transformed into a microparticulate form.

Regardless of the sequence in which these ingredients are added, the form of the active ingredient is in the form of a solution because the incorporation of the solution into the hydrophobic matrix under simultaneous kneading and drying processes results in a homogenous distribution of the active ingredient throughout the matrix. Other forms of active ingredient, such as powder, tend to result in partial phase separation and cannot be used for pharmaceutical purposes. However, these active ingredient forms may function in other technical applications.

In some cases, excess magnesium stearate is added after the matrix is formed. Additionally, the mixture comprising the above-mentioned ingredients is continuously kneaded by pressing and folding the matrix to allow the pharmaceutically active compound to disperse uniformly throughout the matrix. During this process, water is continuously evaporating, and the matrix gradually transforms into microparticulate form. Extra water can be added during this transformation, before obtaining the dry microparticulate form. The final matrix is able to release pharmaceutically active compound to its surroundings in a sustained manner.

Magnesium stearate functions as an anti-caking agent, and the ratio of magnesium stearate to oil determines the properties of the sustained release system. In at least one embodiment, a final matrix contains ten percent pharmaceutically active compound solution in a matrix consisting itself of ninety percent magnesium stearate and ten percent oil. In at least one embodiment, the magnesium stearate, oil, and active ingredient are all biodegradable and biocompatible.

In one embodiment, the final matrix comprises about ten percent weight by volume (w/v) of the pharmaceutically active compound, in a matrix comprising about ninety percent w/v of the anti-caking agent and about ten percent w/v of the oil. It is understood in the art that a one percent weight by volume (w/v) solution refers to 1 gram per 100 milliliters of the solvent. Accordingly, a ten percent w/v of the pharmaceutically active compound refers to a solution that consists of ten grams of pharmaceutically active compound per 100 milliliters of the solvent. This ten percent w/v of pharmaceutically active compound is added to a matrix, which itself comprises about ninety percent w/v of anti-caking agent and about ten percent w/v of oil. In another embodiment the percentages of the anti-caking agent and the oil in the mixture can be varied in such a way that the sum of the percentage w/v of all the components is 100%. For instance, the mixture can comprise about 80% of the anti-caking agent and about 20% of the oil.

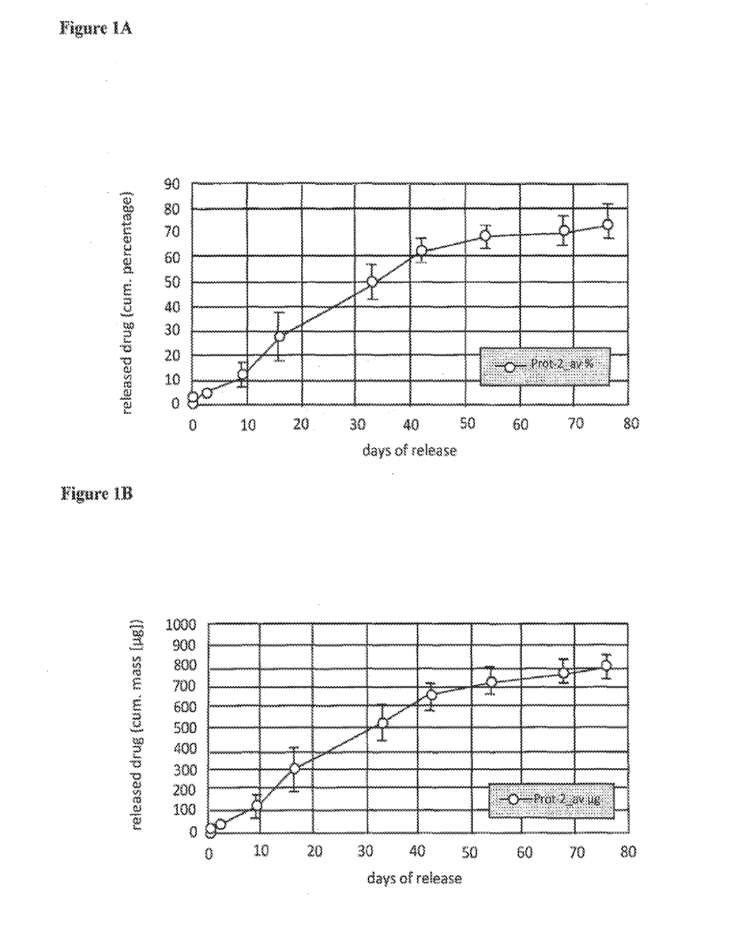

As illustration, the present embodiment discloses two examples of manufacturing the drug-delivery composition discussed herein. Example 1 of the present embodiment discloses a one-step method of manufacturing the drug delivery composition. In this example, tocopherol, magnesium stearate (an anti-caking agent) and protein solution (pharmaceutically active compound dissolved in an aqueous solution) are mixed and kneaded in one step until the mixture is transformed into a microparticulate matrix. It was observed that this drug delivery composition allowed sustained release of the pharmaceutically active compound related to the percentage of the original payload (FIGS. 1A and 1B).

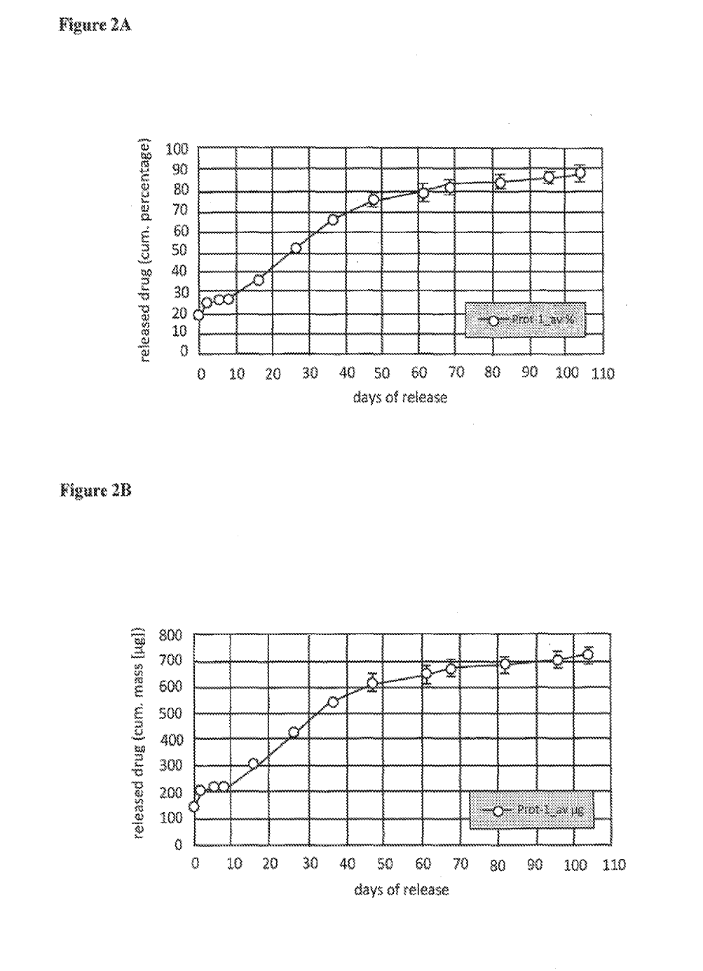

Example 2 of the present embodiment discloses a two-step method of manufacturing the drug delivery composition. In this example, tocopherol and magnesium stearate (anti-caking agent) are mixed and kneaded. The protein solution (pharmaceutically active compound dissolved in aqueous solution) is then added to the mixture and the above ingredients are mixed and kneaded until formation of a dry microparticulate powder. It was observed that this drug delivery composition allowed sustained release of the pharmaceutically active compound related to the percentage of the original payload (FIGS. 2A and 2B).

It is to be further understood that even though numerous characteristics and advantages of the present embodiments have been set forth in the foregoing description, together with details of the structures and functions of the embodiments, the disclosure is illustrative only, and changes may be made in detail, especially in matters of shape, size, and arrangement of parts within the principles of the disclosure to the full extent indicated by the broad general meaning of the terms expressed herein.

### EXAMPLES

The present embodiment is further illustrated by the following examples, which illustrate certain representative embodiments. It is to be understood that the following examples shall not limit the scope of the embodiments in any way.

### Example 1

### One-Step Process of Manufacturing the Drug-Delivery Composition

40 mg of tocopherol and 360 mg of magnesium stearate plus 0.5 ml of protein solution are mixed and kneaded until transformed into a microparticulate matrix. The release profile of this formulation is illustrated in FIGS. 1A-1B. FIG. 1A shows the sustained release of pharmaceutically active compound related to the percentage of original payload of pharmaceutically active compound. FIG. 1B shows the release when normalized to a 10 mg matrix system appropriate for intravitreal injection.

### Example 2

### A Two-Step Process of Manufacturing the Drug Delivery Composition

31 mg of tocopherol and 270 mg of magnesium stearate are mixed and kneaded. Subsequently, 0.15 ml of protein solution is added to the mixture. All ingredients are mixed and kneaded until formation of a dry microparticle powder. The release profile of this formulation is illustrated in FIGS. 2A-2B. FIG. 2A shows the sustained release of pharmaceutically active compound related to the percentage of original payload of pharmaceutically active compound. FIG. 2B shows the release when normalized to a 10 mg matrix system appropriate for intravitreal injection.

While the disclosed embodiments have been particularly shown and described with reference to specific embodiments, it should be understood by those skilled in the art that various changes in form and detail may be made therein without departing from the spirit and scope of the disclosed embodiments as defined by the appended claims. The scope of the disclosed embodiments is thus indicated by the appended claims and all changes which come within the meaning and range of equivalency of the claims are therefore intended to be embraced.

What is claimed is:

1-11. (canceled)

12. A method for manufacturing a drug-delivery composition, comprising:
mixing at least one anti-caking agent and at least one oil, wherein a fraction of said oil is greater than a final concentration of the ready-for-application delivery mix;
adding excess of the anti-caking agent to the mixture comprising the anti-caking agent and the oil;
mixing said anti-caking agent and oil mixture by continuously pressing and folding the mixture to transform the mixture into a microparticulate matrix;
dissolving at least one pharmaceutically active compound in an aqueous solution;
adding said pharmaceutically active compound dissolved in the aqueous solution to the microparticulate matrix; and
dispersing the pharmaceutically active compound dissolved in the aqueous solution within the matrix by continuously pressing and folding the matrix.

13. The method of claim 12, wherein the pharmaceutically active compound comprises a compound selected from the group consisting of a protein, a humanized monoclonal antibody, a human monoclonal antibody, a chimeric antibody, an immunoglobulin, fragment, derivative or fraction thereof, a synthetic, semi-synthetic or biosynthetic substance mimicking immunoglobulins or fractions thereof, an antigen binding protein or fragment thereof, a fusion protein or peptide or fragment thereof, a receptor antagonist, an antiangiogenic compound, an intracellular signaling inhibitor, a peptide with a molecular mass equal to or higher than 3 kDa, a ribonucleic acid (RNA), a deoxyribonucleic acid (DNA), a plasmid, a peptide nucleic acid (PNA), a steroid, a corticosteroid, an adrenocorticostatic, an antibiotic, an antidepressant, an antimycotic, a [beta]-adrenolytic, an androgen or antiandrogen, an antianemic, an anabolic, an anesthetic, an analeptic, an antiallergic, an antiarrhythmic, an antiarterosclerotic, an antibiotic, an antifibrinolytic, an anticonvulsive, an anti-inflammatory drug, an anticholinergic, an antihistamine, an antihypertensive, an antihypotensive, an anticoagulant, an antiseptic, an antihemorrhagic, an antimyasthenic, an antiphlogistic, an antipyretic, a beta-receptor antagonist, a calcium channel antagonist, a cell, a cell differentiation factor, a chemokine, a chemotherapeutic, a coenzyme, a cytotoxic agent, a prodrug of a cytotoxic agent, a cytostatic, an enzyme and its synthetic or biosynthetic analogue, a glucocorticoid, a growth factor, a hemostatic, a hormone and its synthetic or biosynthetic analogue, an immunosuppressant, an immunostimulant, a mitogen, a physiological or pharmacological inhibitor of mitogens, a mineralocorticoid, a muscle relaxant, a narcotic, a neurotransmitter, a precursor of neurotransmitter, an oligonucleotide, a peptide, a (para)-sympathomimetic, a (para)-sympatholytic, a sedating agent, a spasmolytic, a vasoconstrictor, a vasodilator, a vector, a virus, a virus-like particle, a virustatic, a wound healing substance and a combination thereof.

14. The method of claim 12, wherein the pharmaceutically active compound is either alone or in combination with at least one excipient.

15. The method of claim 14, wherein the excipient is selected from the group consisting of monosaccharides, disaccharides, oligosaccharides, polysaccharides, hyaluronic acid, pectin, gum arabic and other gums, albumin, chitosan, collagen, collagen-n-hydroxysuccinimide, fibrin, fibrinogen, gelatin, globulin, polyaminoacids, polyurethane comprising amino acids, prolamin, protein-based polymers, copolymers and derivatives thereof, and mixtures thereof.

16. The method of claim 12, wherein the mixing by repeated pressing and folding comprises repeated cycles of pressing and folding in an algorithmic manner of the matrix and the pharmaceutically active ingredient.

17. The method of claim 12, wherein the mixing by repeated pressing and folding allows water in the matrix to evaporate.

18. The method of claim 12, wherein the pressing comprises applying a pressure of not more than 10⁶N·m⁻².

19. The method of claim 12, wherein the anti-caking agent is a compound selected from the group consisting of magnesium stearate, magnesium palmitate and other similar compounds.

20. The method of claim 12, wherein the oil is selected from the group consisting of plant oil, castor oil, jojoba oil, soybean oil, silicon oils, paraffin oils and mineral oils, and oxethylated plant oils.

21. The method of claim 12, wherein the final matrix comprises about ten percent weight by volume (w/v) of the pharmaceutically active compound, in a matrix comprising preferably about ninety percent w/v of the anti-caking agent and about ten percent w/v of the oil.

22. (canceled)

23. A method for manufacturing a drug-delivery composition, comprising:
dissolving at least one pharmaceutically active compound in an aqueous solution;
mixing said pharmaceutically active compound dissolved in the aqueous solution with at least one anti-caking agent, at least one oil, and water to form a matrix that embeds said pharmaceutically active compound; and
mixing said matrix and said embedded pharmaceutically active compound further until said mixture is transformed into a microparticulate form.

24. The method of claim 23, wherein the pharmaceutically active compound comprises a compound selected from the group consisting of a protein, a humanized monoclonal antibody, a human monoclonal antibody, a chimeric antibody, an immunoglobulin, fragment, derivative or fraction thereof, a synthetic, semi-synthetic or biosynthetic substance mimicking immunoglobulins or fractions thereof, an antigen binding protein or fragment thereof, a fusion protein or peptide or fragment thereof, a receptor antagonist, an antiangiogenic compound, an intracellular signaling inhibitor, a peptide with a molecular mass equal to or higher than 3 kDa, a ribonucleic acid (RNA), a deoxyribonucleic acid (DNA), a plasmid, a peptide nucleic acid (PNA), a steroid, a corticosteroid, an adrenocorticostatic, an antibiotic, an antidepressant, an antimycotic, a [beta]-adrenolytic, an androgen or antiandrogen, an antianemic, an anabolic, an anesthetic, an analeptic, an antiallergic, an antiarrhythmic, an antiarterosclerotic, an antibiotic, an antifibrinolytic, an anticonvulsive, an anti-inflammatory drug, an anticholinergic, an antihistamine, an antihypertensive, an antihypotensive, an anticoagulant, an antiseptic, an antihemorrhagic, an antimyasthenic, an antiphlogistic, an antipyretic, a beta-receptor antagonist, a calcium channel antagonist, a cell, a cell differentiation factor, a chemokine, a chemotherapeutic, a coenzyme, a cytotoxic agent, a prodrug of a cytotoxic agent, a cytostatic, an enzyme and its synthetic or biosynthetic analogue, a glucocorticoid, a growth factor, a hemostatic, a hormone and its synthetic or biosynthetic analogue, an immunosuppressant, an immunostimulant, a mitogen, a physiological or pharmacological inhibitor of mitogens, a mineralocorticoid, a muscle relaxant, a narcotic, a neurotransmitter, a precursor of neurotransmitter, an oligonucleotide, a peptide, a (para)-sympathomimetic, a (para)-sympatholytic, a sedating agent, a spasmolytic, a vasoconstrictor, a vasodilator, a vector, a virus, a virus-like particle, a virustatic, a wound healing substance and a combination thereof.

25. The method of claim 23, wherein the pharmaceutically active compound is either alone or in combination with at least one excipient.

26. The method of claim 25, wherein the excipient is selected from the group consisting of monosaccharides, disaccharides, oligosaccharides, polysaccharides, hyaluronic acid, pectin, gum arabic and other gums, albumin, chitosan, collagen, collagen-n-hydroxysuccinimide, fibrin, fibrinogen, gelatin, globulin, polyaminoacids, polyurethane comprising amino acids, prolamin, protein-based polymers, copolymers and derivatives thereof, and mixtures thereof.

27. The method of claim 23, wherein the mixing of said matrix and said embedded pharmaceutically active compound comprises repeated cycles of pressing and folding in an algorithmic manner of the matrix and the pharmaceutically active ingredient.

28. The method of claim 23, wherein the mixing by repeated pressing and folding allows water in the matrix to evaporate.

29. The method of claim 23, wherein the pressing comprises applying a pressure of not more than 10⁶ N·m⁻².

30. The method of claim 23, wherein the anti-caking agent is a compound selected from the group consisting of magnesium stearate, magnesium palmitate and similar compounds.

31. The method of claim 23, wherein the oil is selected from the group consisting of plant oil, castor oil, jojoba oil, soybean oil, silicon oils, paraffin oils and mineral oils, and oxethylated plant oils.

32. The method of claim 23, wherein the final matrix comprises about ten percent weight by volume (w/v) of the pharmaceutically active compound, in a matrix comprising about ninety percent w/v of the anti-caking agent and about ten percent w/v of the oil.

33-44. (canceled)
